(19) United States
(12) Patent Application Publication
(10) Pub. No.: US 2026/0363813 A1
(43) Pub. Date: Nov. 19, 2026

(54) Intraoperative Organizational Device

(71) Applicant: Timothy Alan Harshman, Atlantic Beach, FL (US)

(72) Inventor: Timothy Alan Harshman, Atlantic Beach, FL (US)

(21) Appl. No.: 19/207,951

(22) Filed: May 14, 2025

(51) Int. Cl. A61B 50/24

(52) U.S. Cl. CPC A61B 50/24

(57) ABSTRACT

The device comprises a top plate comprising a plurality of first openings spaced apart along a length of the top plate and two bottom supports coupled to opposite sides of the top plate. Each bottom support includes having a first end, a second end opposed to the first end, and a waist disposed between the first end and the second end. Each of the two bottom supports extends inwards from the first end to the waist to form an internal space communicated with the plurality of first openings.

### FIELD OF THE DISCLOSURE

The present disclosure relates to the field of intraoperative devices. More specifically, the present disclosure pertains to an intraoperative organizational device that stores and organizes a variety of surgical instruments when they are not being used in a surgical procedure or during the cleaning and sterilization process prior to a surgical procedure.

PRIOR ART

Surgical instruments are essential for surgical procedures including but not limited to orthopedic, neurosurgical, spinal, dental, and laparoscopic types of procedures. Surgical instruments are delicate and available in a variety of sizes and configurations to accommodate a plethora of different surgical needs. Depending on the need for cutting, gouging, or removing bones or tissues, a surgeon or a member of the surgical team may request a surgical technician for the appropriate instrument to perform certain tasks during a surgical procedure. However, if the surgical instruments are not properly organized, several issues may arise. For example, surgical delays may occur when the surgical technician cannot promptly locate the requested or needed surgical instrument. This is commonly due to the lack of organizational devices available to store all of these unique instruments. Furthermore, improperly stowed instruments may come in contact with non-sterile surfaces, or fall from the sterile field, thereby causing a delay in the procedure and increasing the risk of surgical site infection.

Therefore, there is a need for a device to assist with organizing surgical instruments, which not only streamlines access of the instruments, but also mitigates the risk of infection and enhances staff and patient's safety.

### SUMMARY OF THE DISCLOSURE

The following is a concise summary of the disclosure presented herein with the primary aim of providing a preliminary understanding of certain aspects of the disclosure. It should be noted, however, that this summary is not intended to serve as a comprehensive overview of the disclosure, nor does it seek to identify or describe any critical or significant elements of the disclosure or the boundaries of its scope. Its sole purpose is to provide a rudimentary understanding of the disclosure's concepts and features, which will be expounded upon in greater detail in the ensuing sections.

In some embodiments, the device may comprise a top plate comprising a plurality of first openings spaced apart along a length of the top plate; and two bottom supports coupled to opposite sides of the top plate, each bottom support including having a first end, a second end opposed to the first end, and a waist disposed between the first end and the second end; wherein each of the two bottom supports extends inwards from the first end to the waist to form an internal space communicated with the plurality of first openings.

In some embodiments, the device may comprise a top plate comprising a plurality of first openings spaced apart along a length of the top plate; and two bottom supports coupled to opposite sides of the top plate, each bottom support including having a first end, a second end opposed to the first end, and a waist disposed between the first end and the second end; wherein a plurality of second openings corresponding to the plurality of first openings are defined on each of the two bottom supports between the first end of each of the two bottom supports and a bottom end of the waist.

In some embodiments, the device may comprise a top plate comprising a plurality of first openings spaced apart along a length of the top plate; and two bottom supports coupled to opposite sides of the top plate, each bottom support including having a first end and a second end opposed to the first end; wherein each of the two bottom supports extends inwards from the first end to the second end to form an internal space communicated with the plurality of first openings.

The present disclosure pertains to an intraoperative organizational device designed to hold, store and organize surgical instruments or surgical tools, including but not limited to rongeurs, scissors, forceps, and clamps. The disclosed device includes a plurality of spaced-apart first openings configured to receive individual surgical instruments to keep them structurally organized. As surgical instruments come in different lengths and configurations, the disclosed device also comprises a plurality of second openings communicating with the plurality of first openings to accommodate diverse surgical instruments. Notably, the device is integrally formed, avoiding unnecessary crevices that may harbor contamination.

Furthermore, after a surgical tool has been used, a surgical technician may easily replace the used surgical tool within the disclosed device after cleaning any tissue or residue from the jaws of the surgical tool. The device may hold the surgical tools in an open-tip position, minimizing the risk of bone fragments or soft tissue residue remaining in the tips, ensuring the surgical tools are tissue-free when subsequently passed to the surgeon.

Advantageously, the disclosed device not only ensures the surgical tools are being neatly arranged and residue-free, but also improves efficiency of the surgical procedure.

The above features and advantages will become apparent from the following detailed description taken with the accompanying drawings.

### BRIEF DESCRIPTION OF DRAWINGS

FIG. 1 illustrates a first perspective view of a non-limiting exemplary embodiment (i.e., the “exemplary embodiment”) of an intraoperative organizational device (the “device”).

FIG. 2 illustrates a second perspective view of the device of FIG. 1.

FIG. 3 top view of the device of FIG. 1.

FIG. 4 illustrates a right-side view of the device of FIG. 1.

FIG. 5 illustrates a front-side view of the device of FIG. 1.

FIG. 6 illustrates a third perspective view of the device of FIG. 1.

FIG. 7 illustrates a fourth perspective view of the device of claim 1.

FIG. 8 illustrates a bottom view of the device of FIG. 1.

FIG. 9 illustrates an in-use view of the device of FIG. 1.

### NUMBERING REFERENCE

1—Device
10—Top Plate
11—First Openings
19—Main Body
20—Bottom Support
20A—First End
20B—Second End
21—First Upper Section
22—Second Upper Section
23—Waist
24—First Lower Section
25—Second Lower Section
26—Second Openings
30—Surgical Instruments
S-Internal Space

### DETAILED DESCRIPTION OF THE DISCLOSURE

The following detailed description and accompanying drawings provide a comprehensive disclosure of an exemplary embodiment for the purpose of facilitating one of ordinary skill in the relevant art to make and use the disclosure. As such, the detailed description and illustration of the one or more exemplary embodiments presented herein are purely exemplary in nature and are not intended to limit the scope of the disclosure or its protection in any matter. It is further noted that the drawings may not be to scale, and in some cases, certain details may be omitted which are not necessary for an understanding of the present disclosure, such as conventional details of fabrication and assembly. Furthermore, there is no intention to be bound by any expressed or implied theory presented in the preceding technical field, background, brief summary or the following detailed description.

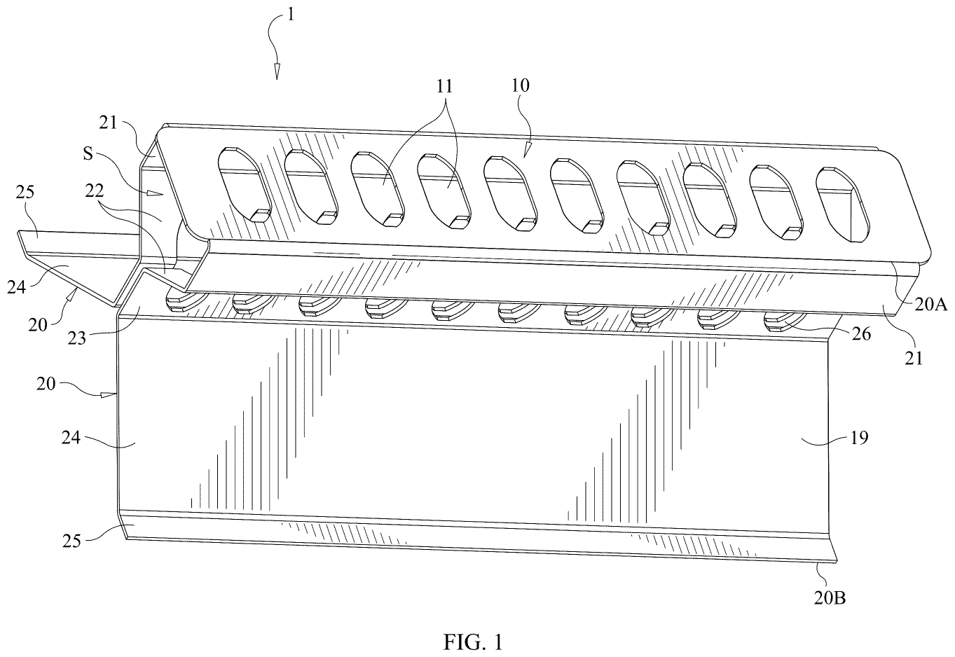

For purposes of description herein, the terms “upper,” “lower,” “front,” “top,” “bottom,” “side” and derivatives thereof shall relate to the device as oriented in FIG. 1. Furthermore, there is no intention to be bound by any expressed or implied theory presented in the preceding technical field, background, brief summary or the following detailed description.

Surgical instruments are designed for a wide variety of surgical procedures and are critical for effective and efficient surgical management. To ensure an aseptic environment and an efficient surgical procedure, it is crucial for the surgical instruments to be well-organized, easily accessible and residue-free after use. The following discloses a device for use in surgical procedures, including but not limited to neurosurgical, orthopedic, ear, nose and throat (ENT), and laparoscopic procedures, and designed to store and organize surgical instruments for quick and easy access upon request.

A non-limiting, exemplary embodiment (herein after as the “exemplary embodiment”) of an intraoperative organizational device 1 (herein after referred to as “the device”) is disclosed herein. The exemplary embodiment of the device 1 may not only keep surgical tools well-organized but can also accommodate surgical tools with different configurations.

FIG. 1 illustrates a first perspective view of the device 1 according to the exemplary embodiment. As shown in FIG. 1, the device 1 comprises a top plate 10 and bottom supports 20 coupled to opposite sides of the top plate 10. FIG. 1 shows two bottom supports 20, although any suitable number of bottom supports is contemplated. The top plate 10, as shown in FIG. 1, has a substantially rectangular shape, although any suitable shape is contemplated. The two bottom supports 20 are preferably coupled to length sides of the rectangular shape of the top plate 10. The two bottom supports 20 can be similarly (and in some examples, identically) configured. For example, each of the two bottom supports 20 has a main body 19 that includes a first end 20A, and a second end 20B opposite the first end 20A. Further, the main body 19 of each of the bottom supports 20 includes a first upper section 21 extending downwards from the first end 20A and a second upper section 22 extending from the first upper section 21. In some embodiments, the bottom portions 20 each include a waist 23. The second upper section 22 extends inwards towards the waist 23. The top plate 10, the first upper section 21, and the second upper section 22 of each of the bottom supports 20 form an internal space S. The waist 23 continues to extend downwards towards the second end 20B. Continuing from the waist 23, a first lower section 24 extends outwards at an angle from the waist 23 and transits to a second lower section 25 that extends horizontally from the first lower section 24. The top plate 10 further comprises a plurality of first openings 11 (ten of which are shown in FIG. 1, although any suitable number of first openings can be included) in communication with the internal space S.

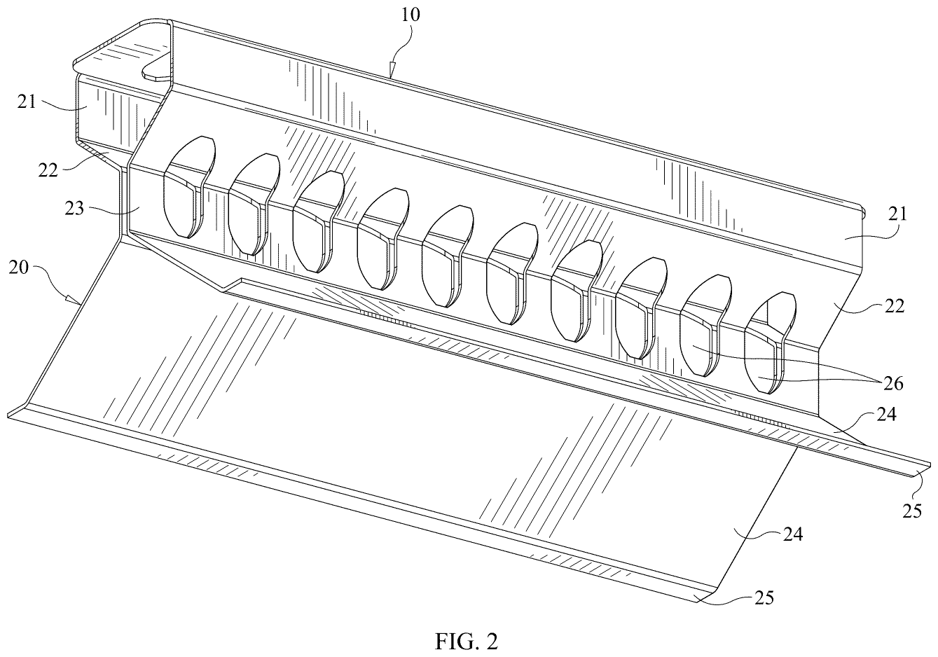

With continuing reference to FIG. 1, FIG. 2 illustrates a second perspective view of the device 1. As shown in FIG. 2, the device 1 further comprises a plurality of second openings 26 (ten of which are shown in FIG. 1, although any suitable number of second openings can be included) defined on each of the two bottom supports 20 between the first end 20A and a bottom end of the waist 23. A number of openings 26 of the plurality of second openings 26 correspond (i.e., is equal to) to a number of openings 11 of the plurality of first openings 11, and are in communication with the internal space S and the plurality of first openings 11.

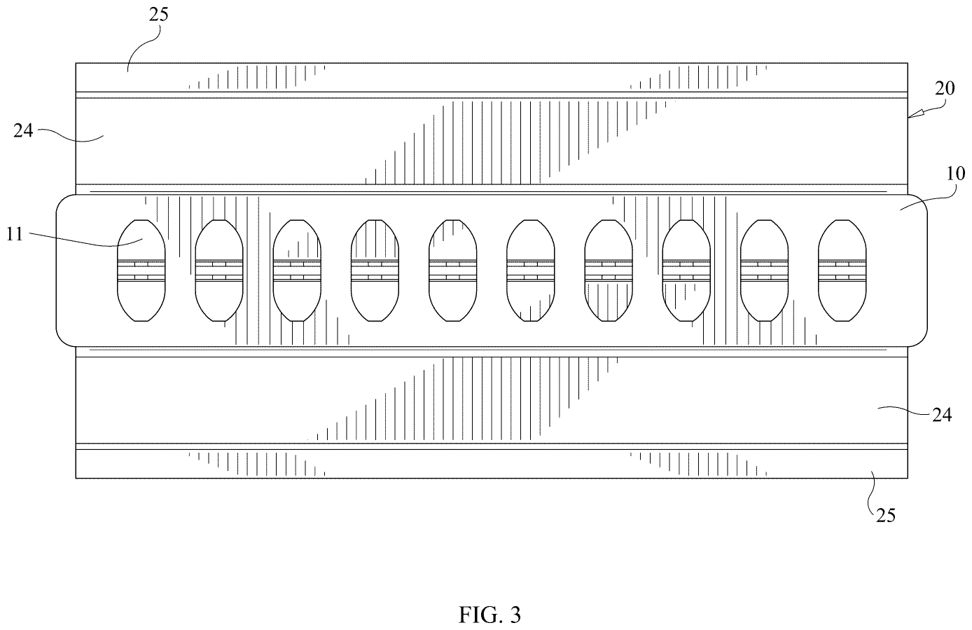

As shown in FIG. 3, FIG. 3 illustrates a top view of the device 1. The plurality of first openings 11 have an elliptical configuration with truncated ends, although any suitable shape for the first openings 11 is contemplated. The plurality of first openings 11 are arranged along the length of the rectangular configuration of the top plate 10 and equally spaced apart, although any suitable spacing arrangement is contemplated.

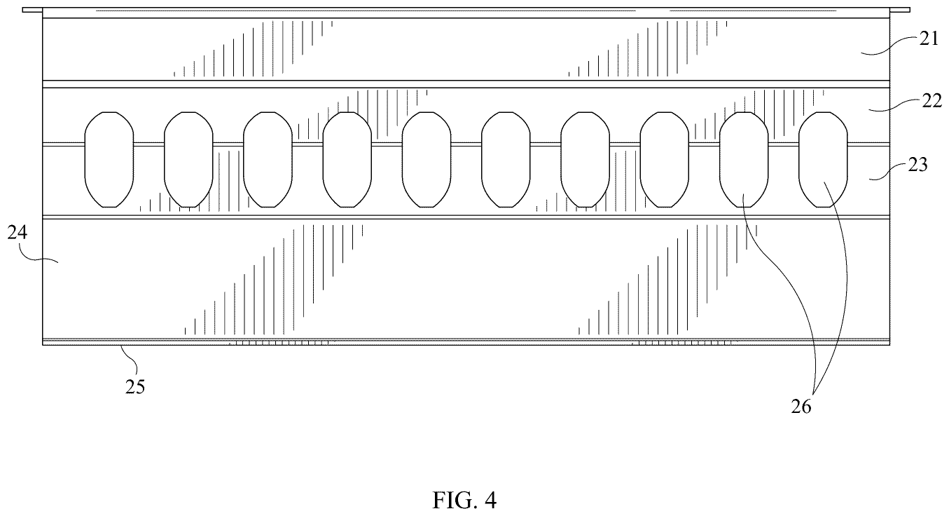

Referring to FIG. 4, the side view of the device 1 shows the plurality of second openings 26. Similar to the plurality of first openings 11, the plurality of second openings 26 have an elliptical configuration with truncated ends, although any suitable shape is contemplated.

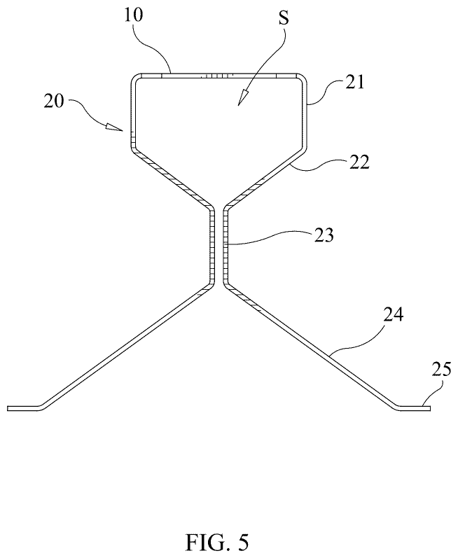

FIG. 5 illustrates a front side view of the device 1. As shown in FIG. 5, the internal space S formed by the top plate 10, the first upper section 21, and the second upper section 22 has a pentagonal cross-sectional shape (although any suitable cross-sectional shape is contemplated). With the second lower sections 25 extending horizontally in opposite directions, the device 1 rests on the two second lower sections 25, evenly distributing the weight of the device 1. Furthermore, the device 1 is integrally formed, which avoids unnecessary crevices that may harbor contamination and eliminates the need for assembly. In addition, the device 1 is preferably constructed from rigid materials that are easy to sterilize, durable and resistant to corrosion, such as stainless steel. It is also anticipated that the device 1 may be constructed from medical/surgical grade plastic for single-use.

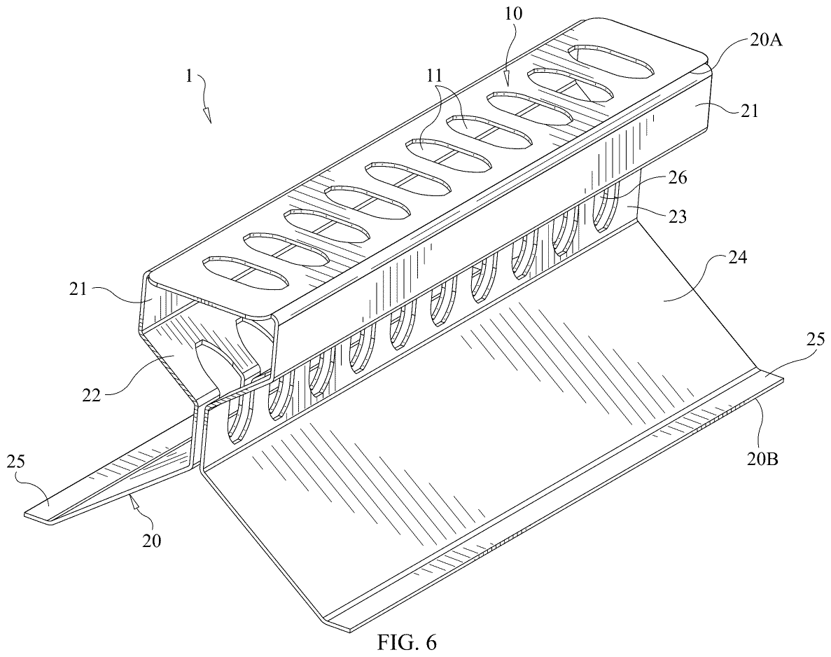

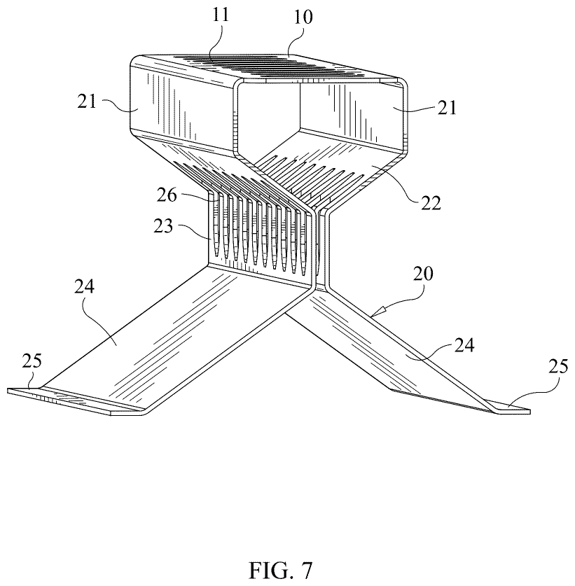

Referring to FIGS. 6 and 7, the plurality of second openings 26 are defined between the first end 20A of each of the two bottom supports 20 and a bottom end of the waist 23. As shown in FIG. 7, the plurality of second openings 26 extend from the second upper section 22 to the waist 23, thereby allowing surgical instruments to migrate to a center of the device 1. Furthermore, the plurality of second openings 26 on one of the two bottom supports 20 aligns with the plurality of second openings 26 on the other bottom support 20 which provides accommodations to surgical instruments with different configurations.

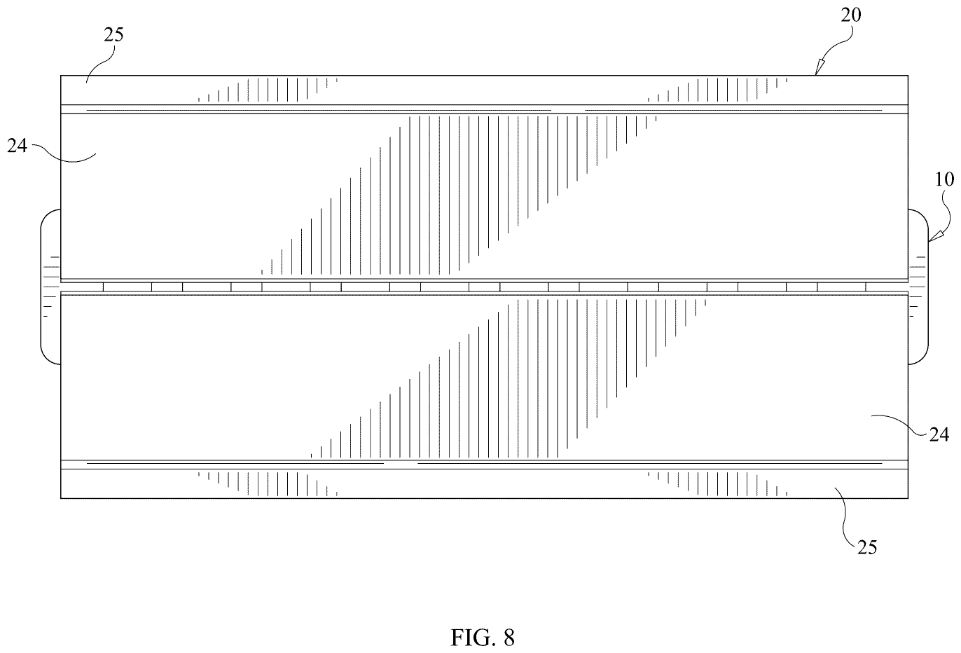

As illustrated in FIG. 8, the bottom view of the device 1 shows that in some embodiments, the length of the top plate 10 may be greater than a length of the two bottom supports 20, although any suitable dimensions are contemplated. Further, a width of the first lower section 24 may be greater than a width of the second lower section 25, although any suitable dimensions are contemplated.

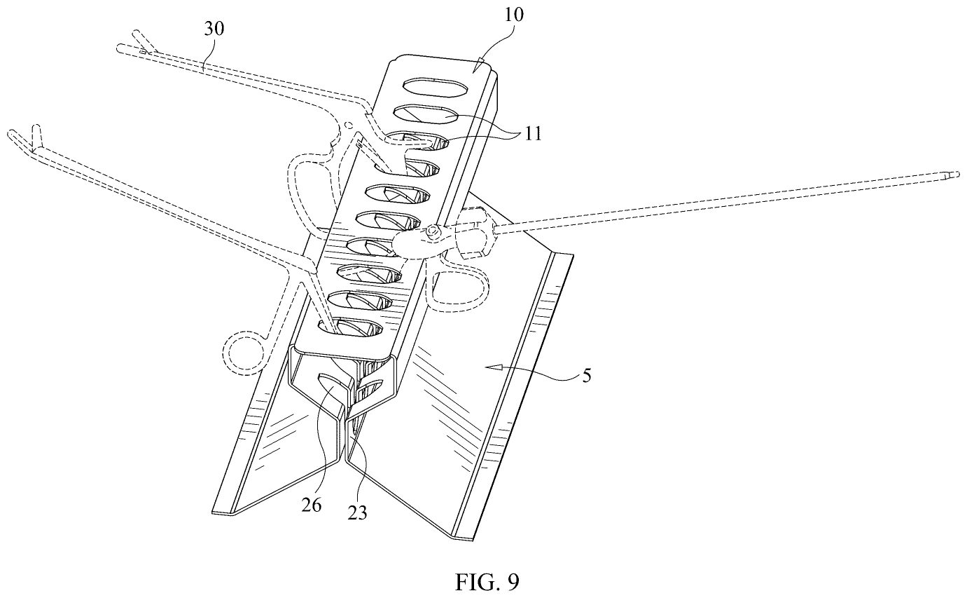

Referring to FIG. 9, an in-use view of the device 1 is presented herein to further demonstrate how surgical instruments are positioned and organized. The device 1 can hold one or more surgical instruments 30 including but not limited to different types of rongeurs and laparoscopic surgical tools based on the need of the surgical procedure. The different types of rongeurs and laparoscopic surgical tools may have handles that are in various forms, such as a levers, rings or loops. As shown in FIG. 9, the one or more surgical instruments 30 are available in various forms and sizes. For example, some surgical instruments 30 have larger finger loops while others have longer or wider handles.

As shown in FIGS. 1 and 2, each of the plurality of the second openings 26 corresponds with one of the plurality of first openings 11, and the first openings 11, the internal space S and the second openings 26 are communicated with each other. Advantageously, the device 1 may accommodate, hold and store surgical instruments 30 with a variety of configurations. For example, the plurality of first openings 11 are sized and dimensioned to receive “smaller” surgical instruments, such as a pituitary type rongeur or a small-ringed laparoscopic instrument, where an end of the surgical instrument that passes through the first opening 11 is disposed in the internal space S. Similarly, the plurality of second openings 26 are sized and dimensioned to receive “larger” surgical instruments where an end of the surgical instrument that passes through the first opening 11 and the internal space S, and is disposed in the second opening 26.

Referring back to FIG. 9, the device 1 receives one of the handles of the surgical instruments 30, such that one of the handles is contained in the internal space S and the other handle is positioned outwards from the device 1, creating a fulcrum for each of the one or more surgical tools to be held by its own weight. As a result, the one or more surgical tools 30, especially non-spring-loaded pituitary type rongeurs, may transit from a closed position to an open position upon full insertion to the device 1. Furthermore, advantageously, a surgical technician may immediately identify the correct requested surgical tool 30 in its upright position and remove any retained tissues from the instrument jaws so that the instruments can be residue and tissue-free upon a next pass to the surgeon. In addition, a user may effortlessly place the handle of the surgical instrument 30 within one of the plurality of first openings 11, and easily locate and reach the needed surgical instrument 30, repeatedly throughout the surgical procedure.

The disclosed device 1 not only offers a convenient, safer and more effective solution for organizing delicate surgical instruments, but also advantageously provides versatility for accommodating surgical instruments with a wide range of configurations used in surgical procedures including but not limited to neurosurgical, orthopedic, ear, nose and throat (ENT), and laparoscopic procedures.

While the embodiments of the disclosure have been disclosed, certain modifications may be made by those skilled in the art to modify the disclosure without departing from the spirit of the disclosure.

What is claimed is:

1. An intraoperative organizational device, the device comprising:
a top plate comprising a plurality of first openings spaced apart along a length of the top plate; and
two bottom supports coupled to opposite sides of the top plate, each bottom support including having a first end, a second end opposed to the first end, and a waist disposed between the first end and the second end;
wherein each of the two bottom supports extends inwards from the first end to the waist to form an internal space communicated with the plurality of first openings.

2. The device of claim 1, further comprising:
a plurality of second openings defined on each of the two bottom supports between the first end of each of the two bottom supports and a bottom end of the waist.

3. The device of claim 2, wherein the plurality of second openings are in communication with the internal space and the plurality of first openings.

4. The device of claim 2, wherein a number of the plurality of second openings is equal to the plurality of first openings.

5. The device of claim 1, wherein the plurality of first openings are equally spaced apart.

6. The device of claim 1, wherein each of the two bottom supports has a first upper section and a second upper section defined between the first end and the waist.

7. The device of claim 6, wherein the first upper section extends downwards from the top plate and the second upper section extends inwards towards the waist.

8. The device of claim 1, wherein each of the two bottom supports has a first lower section and a second lower section defined between the waist and the second end.

9. The device of claim 8, wherein the first lower section inclines downwards at an angle and the second lower section extends horizontally relative to the first lower section.

10. The device of claim 1, wherein each of the two bottom supports extends outwards from the waist to the second end.

11. The device of claim 1, wherein the two bottom supports are identically configured.

12. An intraoperative organizational device, the device comprising:
a top plate comprising a plurality of first openings spaced apart along a length of the top plate; and
two bottom supports coupled to opposite sides of the top plate, each bottom support including having a first end, a second end opposed to the first end, and a waist disposed between the first end and the second end;
wherein a plurality of second openings corresponding to the plurality of first openings are defined on each of the two bottom supports between the first end of each of the two bottom supports and a bottom end of the waist.

13. The device of claim 12, wherein each of the two bottom supports has a first upper section and a second upper section defined between the first end and the waist.

14. The device of claim 13, wherein the first upper section extends downwards from the top plate and the second upper section extends inwards towards the waist.

15. The device of claim 12, wherein each of the two bottom supports has a first lower section and a second lower section defined between the waist and the second end.

16. The device of claim 15, wherein the first lower section inclines downwards at an angle and the second lower section extends horizontally relative to the first lower section.

17. The device of claim 12, wherein the plurality of first openings are equally spaced apart.

18. The device of claim 12, wherein the two bottom supports are identically configured.

19. An intraoperative organizational device, the device comprising:
a top plate comprising a plurality of first openings spaced apart along a length of the top plate; and
two bottom supports coupled to opposite sides of the top plate, each bottom support including having a first end and a second end opposed to the first end;
wherein each of the two bottom supports extends inwards from the first end to the second end to form an internal space communicated with the plurality of first openings.

20. The device of claim 19, further comprising:
a plurality of second openings defined on each of the two bottom supports between the first end and the second end of each of the two bottom supports.
